(19) United States
(12) Patent Application Publication
(10) Pub. No.: US 2003/0147958 A1
(43) Pub. Date: Aug. 7, 2003

(54) Biodegradable multi-block copolymers of poly(amino acid)s and poly(ethylene glycol) for the delivery of bioactive agents

(76) Inventors: Cheol-Hee Ahn, Seoul (KR); Su Young Chae, Gwangju (KR)

Correspondence Address: M. Wayne Western, THORPE NORTH & WESTERN, L.L.P., P.O. Box 1219, Sandy, UT, 84091-1219, US

(21) Appl. No.: 10/061,003

(22) Filed: Jan. 29, 2002

(51) Int. Cl. A61K 48/00; A61K 9/14

(52) U.S. Cl. 424/486; 514/44; 525/54.1

(57) ABSTRACT

This patent discloses the synthesis of a multi-block copolymer containing poly(amino acids) (PAA) and a hydrophilic polymer which are degradable under physiological conditions. Control over the degradation rate of the obtained copolymers is achieved by introducing ester, amide or urethane groups as a biodegradable linkage connecting the PAA and the hydrophilic polymer. The biodegradable multi-block copolymers display high transfection efficiency in plasmid delivery with low cytotoxicity.

### FIELD OF THE INVENTION

This invention relates to the delivery of bioactive agents. More particularly, the invention relates to a composition and method for delivering bioactive agents, such as DNA, RNA, oligonucleotides, proteins, peptides, and drugs, by facilitating their transmembrane transport or by enhancing their adhesion to biological surfaces. It relates particularly to biodegradable multi-block copolymers of a poly(amino acid) (PAA) and a hydrophilic polymer wherein the PAI and the hydrophilic polymer are covalently linked by a biodegradable linkage. The multiblock copolymers of the present invention can be used for drug delivery and are especially useful for delivery of nucleic acids or any anionic bioactive agent.

### BACKGROUND OF INVENTION

Controlled release of bioactive agents can reduce the administration frequency by maintaining the concentration of the therapeutic agent at the desired level, which makes biodegradable delivery systems highly desirable. Biodegradable polymers are gaining attention as drug delivery systems. Jeong et al., Biodegradable Block Copolymers as Injectable Drug-delivery Systems, 388 Nature 860-862 (1997). Delivering bioactive agents from a biodegradable delivery system is highly desirable because the need for a surgical procedure to remove the delivery system is avoided. Controlled release of bioactive agents can reduce the required frequency of administration by maintaining the concentration of the therapeutic agent at desired levels. One important means of maintaining the proper concentration is by controlling the degradation rate of the biodegradable drug delivery system.

Gene therapy is generally considered as a promising approach not only for the treatment of diseases with genetic defects, but also in the development of strategies for treatment and prevention of chronic diseases such as cancer, cardiovascular disease and rheumatoid arthritis. However, nucleic acids, as well as other polyanionic substances are rapidly degraded by nucleases and exhibit poor cellular uptake when delivered in aqueous solutions. Since early efforts to identify methods for delivery of nucleic acids in tissue culture cells in the mid 1950's, steady progress has been made towards improving delivery of functional DNA, RNA, and antisense oligonucleotides in vitro and in vivo.

The gene carriers used so far include viral systems (retroviruses, adenoviruses, adeno-associated viruses, or herpes simplex viruses) or nonviral systems (liposomes, polymers, peptides, calcium phosphate precipitation and electroporation). Viral vectors have been shown to have high transfection efficiency when compared to non-viral vectors, but due to several drawbacks, such as targeting only dividing cells, random DNA insertion, their low capacity for carrying large sized therapeutic genes, risk of replication, and possible host immune reaction, their use in vivo is severely limited.

An ideal transfection reagent should exhibit a high level of transfection activity without the need for any mechanical or physical manipulation of cells or tissues. The reagent should be non-toxic, or minimally toxic, at the effective dose. It should also be biodegradable in order to avoid any long term adverse side effects on the treated cells. When gene carriers are used for delivery of nucleic acids in vivo, it is essential that the gene carriers themselves be nontoxic and that they degrade into non-toxic products. To minimize the toxicity of the intact gene carrier and its degradation products, the design of gene carriers needs to be based on naturally occurring metabolites.

Because of their sub-cellular size, nanoparticles are hypothesized to enhance interfacial cellular uptake, thus achieving in a true sense a “local pharmacological drug effect.” It is also hypothesized that there would be enhanced cellular uptake of drugs contained in nanoparticles (due to endocytosis) compared to the uptake of the corresponding free drug. Nanoparticles have been investigated as drug carrier systems for tumor localization of therapeutic agents in cancer therapy, for intracellular targeting (antiviral or antibacterial agents), for targeting to the reticuloendothelial system (parasitic infections), as immunological adjuvants (by oral and subcutaneous routes), for ocular delivery with sustained drug action, and for prolonged systemic drug therapy.

As compared to viral gene carriers, there are several advantages to the use of non-viral based gene therapies, including their relative safety and low cost of manufacture. Non-viral gene delivery systems such as cationic polymers or synthetic gene carriers, e.g. poly-L-lysine (PLL), are being widely sought as alternatives and are being investigated intensively to circumvent some of the problems encountered with use of viral vectors. J. Cheng et al., Effect of Size and Serum Proteins on Transfection Efficiency of Poly((2-dimethylamino)ethyl methacrylate)-plasmid nanoparticles, 13 Pharm. Res. 1038-1042 (1996). There are several polymeric materials currently being investigated for use as gene carriers, of which poly-L-lysine (PLL) is the most popular, but few of them are biodegradable. Biodegradable polymers, such as polylactic/glycolic acid(negatively charged), and polylactide/glycolide(neutral) have been used as gene carriers in the form of non-soluble particulates. Amarucyama et al, Nanoparticle DNA Carrier with PLL Grafted Polysallanide Copolymer and Polylactic Acid, 8 Bioconjugate, 735-739(1997). In general, polycationic polymers are known to be toxic and the PLL backbone is barely degraded under physiological conditions. It remains in cells and tissues and causes an undesirably high toxicity. A. Segouras & R. Dunlan, Methods for Evaluation of Biocompatibility of Synthetic Polymers, 1 J. Mater.Sci in Medicine, 61-68(1990).

Protamines and histones that contain a high portion of positively charged side groups, such as lysine and arginine, have been known to play a role in condensation and control of expression of DNA in living organisms. Consequently, synthetic poly(amino acid)s have been widely used as carriers for the delivery of bioactive agents. Poly(L-lysine) (PLL) has a number of primary amines with positive charges that interact with the negatively charged phosphate groups of DNA and has been reported to condense plasmid DNA under physiological conditions. PLL displays dependence of cytotoxicity and transfection efficiency on molecular weight, which means that higher transfection efficiency is obtained as the molecular weight of PLL increases, however, at the same time, employment of higher molecular weight PLL leads to enhanced toxicity to cells, S. Choksakulnimitr et al., 34 J. Controlled Release 233 (1995); M. A. Wolfert et al., 3 Gene Ther. 269 (1996). It was reported that PLL with a degree of polymerization less than 5 is not effective in forming stable complexes with DNA, while PLL with a degree of polymerization exceeding 40 shows cytotoxicity, J. G. Duguid et al. 76 Biophys. J. A135 (1996).

In addition, like most cationic polymers, PLL/DNA complexes have drawbacks including precipitation as insoluble particles and the tendency to aggregate into larger complexes under physiological conditions, A. V. Kabanov et al., 6 Bioconjugate Chem. 7 (1995). Proposed approaches to overcome the problems of PLL/DNA complexes are based on the synthesis of copolymers containing PLL and non-ionic hydrophilic segments. Many approaches have been addressed by use of either block or graft copolymers containing hydrophilic segments, in particular, poly (ethylene glycol), A. V. Kabanov et al. 6 Bioconjugate Chem. 639 (1995); S. Katayose et al. 8 Bioconjugate Chem. 702 (1997); Y. H. Choi et al. 54 J. Controlled Release 39 (1998).

PLL does not induce or facilitate the endosomal release of DNA and it limits the transfection efficiency of a gene delivery carrier based on PLL. The primary amino groups in PLL have been utilized to connect to a targeting ligand or endosomal escape moiety thus giving multi-functional capabilities. Introduction of a compound or a polymer with buffering capacities between 4.0 and 7.2 has been reported to enhance the transfection efficiency of PLL, J. M. Benns et al. 11 Bioconjugate Chem. 637 (2000); D. Pack et al. 67 Biotechnol. Bioeng. 217 (2000); D. Putnam et al. 98 Proc. Natl. Acad. Sci. USA 1200 (2001).

In view of the foregoing, development of a gene carrier for gene therapy and drug delivery that is non-toxic, biodegradable, and capable of forming nanoparticles, or transfection complexes will be appreciated and desired. The novel bioactive agent carrier of the present invention comprises a biodegradable multi-block copolymer of a poly(amino acid) (PAA) and a hydrophilic polymer wherein the PAA and the hydrophilic polymer are covalently bound by a biodegradable linkage. The biodegradable multi-block copolymer of the present invention is useful for drug delivery, especially for delivery of nucleic acids, other anionic bioactive molecules, or both, and is readily susceptible to metabolic degradation after incorporation into the cell.

### BRIEF SUMMARY OF THE INVENTION

The present invention provides a biodegradable water soluble multi-block copolymer having reduced in vivo and in vitro toxicity that is useful for delivery of drugs or other bioactive agents to an individual in need thereof.

The present invention also provides biodegradable water soluble multi-block copolymers that are able to condense DNA and form stable complexes with DNA under physiological conditions.

The present invention further provides an efficient non-viral polymer-based system for delivery of DNA or RNA to a target cell.

The present invention further provides an efficient synthesis method to introduce an endosomal escape moiety into the copolymer thereby enhancing the transfection efficiency.

The biodegradable multi-block copolymer of the present invention comprises a poly(amino acid) (PAA) and a hydrophilic polymer wherein the PAA and the hydrophilic polymer are covalently bound by a biodegradable linkage. Preferably, the PAAs of the present invention contain a high portion of positively charged side groups such as polylysine and polyarginine. Preferably, the hydrophilic polymer is a member selected from the group consisting of polyethylene glycol (PEG), polypropylene glycol, poloxamers, poly(acrylic acid), poly(styrene sulfonate), carboxymethylcellulose, poly(vinyl alcohol), polyvinylpyrrolidone, alpha-substituted poly(oxyalkyl) glycols, poly(oxyalkyl) glycol copolymers and block copolymers, and activated derivatives thereof. More preferably, the hydrophilic polymer is a member selected from the group consisting of polyethylene glycol (PEG), polypropylene glycol, poloxamers, poly(acrylic acid), poly(styrene sulfonate), carboxymethylcellulose, poly(vinyl alcohol) and polyvinylpyrrolidone. The most preferred hydrophilic polymer is polyethylene glycol (PEG).

The PAAs of the present invention, polylysine and polyarginine, contain positively charged primary amino groups in each repeating unit under physiological conditions. Optimization of the balance between polymer cationic density and the endosomal escape moiety provides for effective gene transfer with low cytotoxicity and high transfection efficiency. Introduction of endosomal escape moieties such as imidazole derivatives, histidine derivatives, poly(ethylenimine) and poly(L-histidine) with buffering capacities between 4.0 and 7.2 to the primary amino groups in PAA is expected to enhance the transfection efficiency of the biodegradable multi-block copolymer of the present invention. The PAA is conjugated to the hydrophilic polymer by a biodegradable linkage which can be an ester, amide or urethane, depending on the required degradation rate. The molar ratio of the PAA to the hydrophilic polymer is preferably within a range of 0.1 to 2. A preferred cationic copolymer is a copolymer of a low molecular weight PAA and PEG, which exhibits negligible toxicity and high transfection efficiency.

The biodegradable multi-block copolymers can be synthesized by polymerizing N-carboxy-α-amino acid anhydride and hydrophilic blocks. The polymerization mechanism of N-carboxy-α-amino acid anhydride strongly depends on the nature of the initiator. When a nucleophilic initiator, such as a primary amine is employed, polymerization proceeds to yield a polymer with an amino group at one end and an incorporated initiator at the other end. Following the same mechanism, polymerization by an initiator containing a primary amine at both ends, e.g. an alkyldiamine, produces a polymer with an amino group at each end of the polymer chain. These amino groups can be further utilized for reaction with difunctional hydrophilic polymers to produce a multi-block copolymer and for the introduction of a biodegradable linkage.

The cationic multi-block copolymers of the present invention can spontaneously form discrete nanometer-sized particles with a nucleic acid, which can promote more efficient gene transfection into mammalian cells and show reduced cell toxicity. The multi-block copolymer of the present invention is readily susceptible to metabolic degradation after incorporation into animal cells. Moreover, the water soluble cationic multi-block copolymer can form an aqueous micellar solution which is particularly useful for systemic delivery of various bioactive agents such as DNA, proteins, hydrophobic or hydrophilic drugs. The water insoluble multi-block copolymer can form cationic nanoparticles which is particularly useful for local drug delivery. Therefore, the biocompatible and biodegradable cationic multi-block copolymer of this invention provides an improved gene carrier for use as a general reagent for transfection of mammalian cells, and for the in vivo application of gene therapy.

The present invention further provides transfection formulations comprising a novel cationic copolymer complexed with a selected nucleic acid in the proper charge ratio(positve charge of the copolymer/negative charge of the nucleic acid), that is optimally effective for both in vivo and in vitro transfection. Particularly, the weight ratio of DNA to the cationic block copolymer is preferably within a range of 1:0.3 to 1:16.

This invention also provides for a method of transfecting a cell in vitro with biodegradable water soluble multi-block copolymers and a selected plasmid DNA, comprising the steps of:

(a) providing a composition comprising a complex with an effective amount of positively charged biodegradable multi-block copolymers and plasmid DNA.

(b) contacting the cell with an effective amount of the composition such that the cell internalizes the selected plasmid DNA; and

(c) culturing the cell with the internalized selected plasmid DNA under conditions favorable for the growth thereof.

### BRIEF DESCRIPTION OF THE DRAWINGS

FIG. 1 illustrates the reaction mechanism of NCA polymerization and the synthetic scheme of biodegradable water soluble multi-block copolymers from PAA and difunctional PEG.

FIG. 2 illustrates GPC traces of polymers before and after the addition of a difunctional PEG.

FIG. 3 shows agarose gel electrophoresis of a marker gene, pSV-β-gal plasmid, and a copolymer comprised of PLL (degree of polymerization, 26) and PEG (molecular weight, 1,500) at various copolymer/plasmid charge ratios.

FIG. 4 shows the zeta potential measurement of complexes formed by a copolymer comprised of PLL (degree of polymerization, 26) and PEG (molecular weight, 1,500) and plasmid DNA.

FIG. 5 illustrates the estimation of complex sizes for a copolymer comprised of PLL (degree of polymerization, 26) and PEG (molecular weight, 1,500) and plasmid DNA.

FIG. 6 illustrates the cytotoxicity evaluation of copolymers with different charge ratios of a comprised of PLL (degree of polymerization, 26) and PEG (molecular weight, 1,500) on 293T cells by an MTT assay.

FIG. 7 shows the β-galactosidase activity of the complexes on 293T cells by a copolymer comprised of PLL (degree of polymerization, 26) and PEG (molecular weight, 1,500) and plasmid DNA with different charge ratios.

FIG. 8 shows degradation of the synthesized copolymer in PBS buffer at 37° C. as a function of time.

FIG. 9 shows representative GPC traces of the degraded copolymer.

FIG. 10 shows the transfection efficiency of the copolymer in the presence and absence of serum.

FIG. 11 illustrates the cytotoxicity of the copolymer conjugated with different amounts of a n endosomal escape moiety.

FIG. 12 illustrates the transfection efficiency of the copolymer with different amount of an endosomal escape moiety.

FIG. 13 illustrates the transfection efficiency of the conjugated copolymer in the presence and absence of serum.

### DETAILED DESCRIPTION OF THE INVENTION

Before the present composition and method for delivery of a bioactive agent are disclosed and described, it is to be understood that this invention is not limited to the particular configurations, process steps, and materials disclosed herein as such configurations, process steps, and materials may vary somewhat. It is also to be understood that the terminology employed herein is used for the purpose of describing particular embodiments only and is not intended to be limiting since the scope of the present invention will be limited only by the appended claims and equivalents thereof.

It must be noted that, as used in this specification and the appended claims, the singular forms “a,” “an,” and “the” include plural referents unless the context clearly dictates otherwise. Thus, for example, reference to a polymer containing “a sugar” includes reference to two or more of such sugars, reference to “a ligand” includes reference to one or more of such ligands, and reference to “a drug” includes reference to two or more of such drugs.

In describing and claiming the present invention, the following terminology will be used in accordance with the definitions set out below.

“Transfecting” or “transfection” shall mean transport of nucleic acids from the environment external to a cell to the internal cellular environment, with particular reference to the cytoplasm and/or cell nucleus. Without being bound by any particular theory, it is understood that nucleic acids may be delivered to cells either after being encapsulated within or adhering to one or more cationic lipid/nucleic acid complexes or entrained therewith. Particular transfecting instances deliver a nucleic acid to a cell nucleus. Nucleic acids include both DNA and RNA as well as synthetic congeners thereof. Such nucleic acids include missense, antisense, nonsense, as well as protein producing nucleotides, on and off and rate regulatory nucleotides that control protein, peptide, and nucleic acid production. In particular, but nonlimiting, they can be genomic DNA, cDNA, mRNA, tRNA, rRNA, hybrid sequences or synthetic or semi-synthetic sequences, and of natural or artificial origin. In addition, the nucleic acid can be variable in size, ranging from oligonucleotides to chromosomes. These nucleic acids may be of human, animal, vegetable, bacterial, viral, and the like, origin. They may be obtained by any technique known to a person skilled in the art.

As used herein, the term “bioactive agent” or “drug” or any other similar term means any chemical or biological material or compound suitable for administration by methods previously known in the art and/or by the methods taught in the present invention and that induce a desired biological or pharmacological effect, which may include but is not limited to (1) having a prophylactic effect on the organism and preventing an undesired biological effect such as preventing an infection, (2) alleviating a condition caused by a disease, for example, alleviating pain or inflammation caused as a result of disease, and/or (3) either alleviating, reducing, or completely eliminating a disease from the organism. The effect may be local, such as providing for a local anaesthetic effect, or it may be systemic.

This invention is not drawn to novel drugs or to new classes of bioactive agents per se. Rather it is drawn to biodegradable cationic multi-block copolymer compositions and methods of using such compositions for the delivery of genes or other bioactive agents that exist in the state of the art or that may later be established as active agents and that are suitable for delivery by the present invention. Such substances include broad classes of compounds normally delivered into the body. In general, this includes but is not limited to: nucleic acids, such as DNA, RNA, and oligonucleotides; antiinfectives such as antibiotics and antiviral agents; analgesics and analgesic combinations; anorexics; antihelminthics; antiarthritics; antiasthmatic agents; anticonvulsants; antidepressants; antidiabetic agents; antidiarrheals; antihistamines; antiinflammatory agents; antimigraine preparations; antinauseants; antineoplastics; antiparkinsonism drugs; antipruritics; antipsychotics; antipyretics; antispasmodics; anticholinergics; sympathomimetics; xanthine derivatives; cardiovascular preparations including potassium, calcium channel blockers, beta-blockers, alpha-blockers, and antiarrhythmics; antihypertensives; diuretics and antidiuretics; vasodilators including general, coronary, peripheral and cerebral; central nervous system stimulants; vasoconstrictors; cough and cold preparations, including decongestants; hormones such as estradiol and other steroids including corticosteroids; hypnotics; immunosuppressives; muscle relaxants; parasympatholytics; psychostimulants; sedatives; and tranquilizers. By the method of the present invention, drugs in all forms, e.g. ionized, nonionized, free base, acid addition salt, and the like may be delivered, as can drugs of either high or low molecular weight. The only limitation to the genus or species of bioactive agent to be delivered is that of functionality, which can be readily determined by routine experimentation.

As used herein, the term “biodegradable” or “biodegradation” is defined as the conversion of materials into less complex intermediates or end products by solubilization hydrolysis, or by the action of biologically formed entities which can be enzymes and other products of the organism.

As used herein, “effective amount” means an amount of a nucleic acid or a bioactive agent that is sufficient to provide the desired local or systemic effect and performance at a reasonable risk/benefit ratio as would attend any medical treatment.

As used herein, “peptide”, means peptides of any length and includes proteins. The terms “polypeptide” and “oligopeptide” are used herein without any particular intended size limitation, unless a particular size is otherwise stated. Typical of peptides that can be utilized are those selected from the group consisting of oxytocin, vasopressin, adrenocorticotrophic hormone, epidermal growth factor, prolactin, luliberin or luteinising hormone releasing hormone, growth hormone, growth hormone releasing factor, insulin, somatostatin, glucagon, interferon, gastrin, tetragastrin, pentagastrin, urogastroine, secretin, calcitonin, enkephalins, endorphins, angiotensins, renin, bradykinin, bacitracins, polymixins, colistins, tyrocidin, gramicidines, and synthetic analogues, modifications and pharmacologically active fragments thereof, monoclonal antibodies and soluble vaccines. The only limitation to the peptide or protein drug which may be utilized is one of functionality.

As used herein, a “derivative” of a carbohydrate includes, for example, an acid form of a sugar, e.g. glucuronic acid; an amine of a sugar, e.g. galactosamine; a phosphate of a sugar, e.g. mannose-6-phosphate; and the like.

As used herein, “administering”, and similar terms means delivering the composition to the individual being treated such that the composition is capable of being circulated systemically where the composition binds to a target cell and is taken up by endocytosis. Thus, the composition is preferably administered to the individual systemically, typically by subcutaneous, intramuscular, transdermal, intravenous, or intraperitoneal administration. Injectables for such use can be prepared in conventional forms, either as a liquid solution or suspension, or in a solid form that is suitable for preparation as a solution or suspension in a liquid prior to injection, or as an emulsion. Suitable excipients that can be used for administration include, for example, water, saline, dextrose, glycerol, ethanol, and the like; and if desired, minor amounts of auxiliary substances such as wetting or emulsifying agents, buffers, and the like.

Development of a safe and efficient gene delivery carrier is an important factor to the success of gene therapy. Poly(amino acid)s (PAA) have been proven to be effective in gene transfer. However, the degradation rate of PAA under physiological conditions is slow and their complexes with plasmid DNA have drawbacks, including precipitation as insoluble particles and the tendency to aggregate into larger complexes under physiological conditions. In addition, the effectiveness as a gene delivery carrier is dependent on their molecular weight and charge ratios to plasmid DNA. High molecular weight PAAs with a degree of polymerization exceeding 40 are sufficiently toxic to the cells and tissues to render them not useful. Low molecular weight PAAs with a degree of polymerization less than 5 are less toxic, but their transfection efficiency is not sufficient, probably due to the formation of unstable complexes with plasmid DNA. The problems caused by their molecular weight dependence and slow degradation rate are expected to be overcome, as presented in this invention, by a multi-block copolymer comprised of low molecular weight PAAs showing low cytotoxicity, and a hydrophilic polymer connected by a biodegradable linkage whose degradation rate is adjustable.

Polymerization of N-carboxy-α-amino acid anhydride, protected at the ε position by a benzyloxycarbonyl group, with an initiator containing a primary amine at both ends produces a poly (amino acid) (PAA) with a primary amino group at each end of the polymer chain. The difunctional PEGs used in the present invention are derivatives of PEG bearing electrophilic groups and are reactive towards the primary amino groups at the end of the PAA and produce a multi-block copolymer. The chemical structure of the multi-block copolymer in the present invention can be simplified as follows

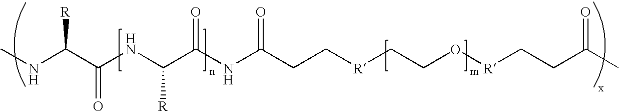

wherein n is an integer from 5 to 1,000, m is an integer from 10 to 500, x is an integer from 1 to 100, R′ represent a biodegradable linkage, and R represents the residual portion of an amino acid or derivatives thereof. Preferably, R′ is a linkage member selected from the group consisting of ester, amide, urethane and carbonate and more preferably is R′ is a linkage member selected from the group consisting of ester, amide, urethane. Preferably, and R represents the residual portion of an amino acid has positively charged side chains under a physiological condition such as lysine, arginine or derivatives thereof.

In accordance with the present invention, the biodegradable linkage, R′, can be prepared as an ester, amide or urethane, depending on the nature of the chemical structure connecting the hydroxysuccinimidyl group and the PEG backbone. This variability in selecting the linkage group is believed very useful because we can selectively synthesize copolymers displaying different degradation rates depending on the nature of the linkage group.

Preferably, the PAAs of the present invention contain a high portion of positively charged side groups such as polylysine and polyarginine. The hydrophilic polymer covalently connected to the poly(amino acid)s by a biodegradable linkage is a member selected from the group consisting of polyethylene glycol (PEG), poloxamers, poly(acrylic acid), poly(styrene sulfonate), carboxymethylcellulose, poly(vinyl alcohol), polyvinylpyrrolidone, alpha-substituted poly(oxyalkyl) glycols, poly(oxyalkyl) glycol copolymers and block copolymers, and activated derivatives thereof. The most preferred hydrophilic polymer is polyethylene glycol (PEG). Preferably, the average molecular weight of the PAA is within a range of 800 to 1,000,000 Daltons and the average molecular weight of the hydrophilic polymer is within a range of 500 to 20,000 Daltons. The PAA is conjugated to the hydrophilic polymer by a biodegradable linkage which can be an ester, amide or urethane, depending on the required degradation rate. The molar ratio of the PAA to the hydrophilic polymer is preferably within a range of 0.5 to 2. A preferred multi-block copolymer is a copolymer of a low molecular weight PAA and PEG, which exhibits negligible toxicity and high transfection efficiency.

Hydrophilic PEG is expected to reduce the toxicity of the copolymer, improve the poor solubility of the PAA and DNA complexes, and help to introduce biodegradable groups by reaction with the primary amines in the both ends of the PAA. Considering the dependence of transfection efficiency and cytotoxicity on the molecular weight of the PAA, high transfection efficiency is expected from an increased molecular weight of the copolymer and low cytotoxicity from the degradation of the copolymer into minimally toxic low molecular weight PAAs.

The PAAs of the present invention, polylysine and polyarginine, contain positively charged primary amino groups in each repeating unit under physiological conditions and do not induce or facilitate the endosomal release of DNA. Optimization of the balance between the polymer cationic density and the endosomal escape moiety has been investigated for effective gene transfer with low cytotoxicity and high transfection efficiency. Endosomal escape moieties help polymer-DNA complexes escape from the endosomes and thus enhance the transfection efficiency. It has been attributed to so-called proton-sponge effect hypothesis, namely that unprotonated moieties on the polymer can buffer the pH inside the endocytic vesicle. In addition, the hypothesis states that influx of counter-ions, which are brought into the vesicle in order to maintain electroneutrality, induces osmotic swelling and rupture of the vesicle membrane. Introduction of endosomal escape moieties such as imidazole derivatives, histidine derivatives, poly(ethylenimine) and poly(L-histidine) with buffering capacities between 4.0 and 7.2 to the primary amino groups in PAA is expected to enhance the transfection efficiency of the biodegradable multi-block copolymer of the present invention.

The cationic copolymers of the present invention can spontaneously form discrete nanometer-sized particles with a nucleic acid, which can promote more efficient gene transfection into mammalian cells and show reduced cell toxicity. The copolymer of the present invention is readily susceptible to metabolic degradation after incorporation into animal cells. Moreover, the multi-block copolymer can form an aqueous micellar solution which is particularly useful for the systemic delivery of various bioactive agents. Therefore, the biocompatible and biodegradable multi-block copolymer of this invention provides an improved gene carrier for use as a general reagent for transfection of mammalian cells, and for the in vivo application of gene therapy.

The present invention further provides transfection formulations, comprising a novel multi-block copolymer complexed with a selected nucleic acid, in the proper charge ratio (positive charge of the copolymer/negative charge of the nucleic acid), that is optimally effective for both in vivo and in vitro transfection. Particularly, the charge ratio of DNA to the cationic copolymer is preferably within a range of 1:1 to 1:10.

The multi-block copolymer of the present invnetion can also be conjugated, either directly or via spacer molecules, with targeting ligands. The target ligands conjugated to the multi-block copolymer direct the copolymer-nucleic acid/drug complex to bind to specific target cells and penetrate into such cells(tumor cells, liver cells, heamatopoietic cells, and the like). The target ligands can also be an intraellular targeting element, enabling the transfer of the nucleic acid/drug to be guided towards certain favored cellular compartments (mitochondria, nucleus, and the like). In a preferred embodiment, the ligands can be sugar moieties coupled to the amino groups. Such sugar moieties are preferably mono- or oligo-saccharides, such as galactose, glucose, fucose, fructose, lactose, sucrose, mannose, cellobiose, nytrose, triose, dextrose, trehalose, maltose, galactosamine, glucosamine, galacturonic acid, glucuronic acid, and gluconic acid. The galactosyl unit of lactose provides a convenient targeting molecule for hepatocyte cells because of the high affinity and avidity of the galactose receptor on these cells.

Other types of targeting ligands that can be used include peptides such as antibodies or antibody fragments, cell receptors, growth factor receptors, cytokine receptors, transferrin, epidermal growth factor (EGF), insulin, asialoorosomucoid, mannose-6-phosphate (monocytes), mannose (macrophage, some B cells), Lewis^{X} and sialyl Lewis^{X} (endothelial cells), N-acetyllactosamine (T cells), galactose (colon carcinoma cells), and thrombomodulin (mouse lung endothelial cells), fusogenic agents such as polymixin B and hemaglutinin HA2, lysosomotrophic agents, nucleus localization signals (NLS) such as T-antigen, and the like.

An advantage of the present invention is that it provides a gene carrier wherein the particle size and charge density are easily controlled. Control of particle size is crucial for optimization of a gene delivery system because the particle size often governs the transfection efficiency, cytotoxicity, and tissue targeting in vivo. In general, in order to enable its effective penetration into tissue, the size of a gene delivery particle should not exceed the size of a virus. In the present invention, the particle size can be varied by using different ratios of the PAA to PEG and by varying the initial molecular weight of the PAA and PEG, which in turn determines the particle size of the-nucleic acid complex.

In a preferred embodiment of the invention, the particle sizes will range from about 80 to 200 nm depending on the cationic copolymer composition and the mixing ratio of the components. It is known that particles, nanospheres, and microspheres of different sizes, when injected, accumulate in different organs of the body depending on the size of the particles injected. For example, after systemic administration, particles of less than 150 nm diameter can pass through the sinusoidal fenestrations of the liver endothelium and become localized, in the spleen, bone marrow, and possibly tumor tissue. Intravenous, intra-arterial, or intraperitoneal injection of particles approximately 0.1 to 2.0 μm diameter leads to rapid clearance of the particles from the blood stream by macrophages of the reticuloendothelial system.

It is believed that the presently claimed composition is effective in delivering, by endocytosis, a selected nucleic acid into hepatocytes mediated by galactosyl receptors on the surface of the hepatocyte cells. Nucleic acid transfer to other cells can be carried out by matching a cell having a selected receptor thereof with a selected sugar. For example, the carbohydrate-conjugated cationic lipids of the present invention can be prepared from mannose for transfecting macrophages, from N-acetyllactosamine for transfecting T cells, and galactose for transfecting colon carcinoma cells.

Since cationic copolymers are known to be good for intracellular delivery of substances other than nucleic acids, the biodegradable multiblock copolymers of PAA and PEG can be used for the cellular delivery of substances other than nucleic acids, such as, for example, proteins and various pharmaceutical or bioactive agents. Examples of peptide and protein drugs include, but are not limited to LHRH analogues, desmopressin, oxytocin, neurotensin, acetylneurotensin, captopril, carbetocin, antocin II, octreotide, thyrotropin-releasing hormnone(TRH), cyclosporine, enkephalins, insulin, calcitonin, interferons, GM-CSF, G-CSF, alpha-1 antitrpsin, alpha-a proteinase inhibitor, dexoyribonuclease, growth hormone, growth factors, and erythropoietin.

The present invention therefore provides methods for treating various disease states, so long as the treatment involves transfer of material into cells. In particular, treating the following disease states is included within the scope of this invention: cancers, infectious diseases, inflammatory diseases and genetic hereditary diseases.

The biodegradable multi-block copolymers of a PAA and a hydrophilic polymer, as described herein, exhibit improved cellular binding and uptake characteristics toward the bioactive agent to be delivered. As such, the present invention overcomes the problems as set forth above. For example, the biodegradable cationic copolymer of the PAA and PEG is easily hydrolyzed or converted to a low molecular weight PAA and PEG in the body. The degraded low molecular weight PAA and PEG will easily be eliminated from the body. In addition, the degradation products are small, non-toxic molecules that are subject to renal excretion and are inert during the period required for gene expression. Degradation is by simple hydrolytic and/or enzymatic reaction. Enzymatic degradation may be significant in certain organelles, such as lysosomes. It is particularly advantageous for the present invention that the degradation rate of the multi-block copolymer can be controlled by choosing different biodegradable linkages between the PAA and PEG.

Furthermore, nanoparticles or transfection complexes can be formed from the cationic copolymer and nucleic acids or other negatively charged bioactive agents by simple mixing. Therefore, the cationic gene carrier of the present invention provides improved transfection efficiency and reduced cell toxicity.

The following examples will enable those skilled in the art to more clearly understand how to practice the present invention. It is to be understood that, while the invention has been described in conjunction with the preferred specific embodiments thereof, that which follows is intended to illustrate and not limit the scope of the invention. Other aspects of the invention will be apparent to those skilled in the art to which the invention pertains.

### EXAMPLE 1

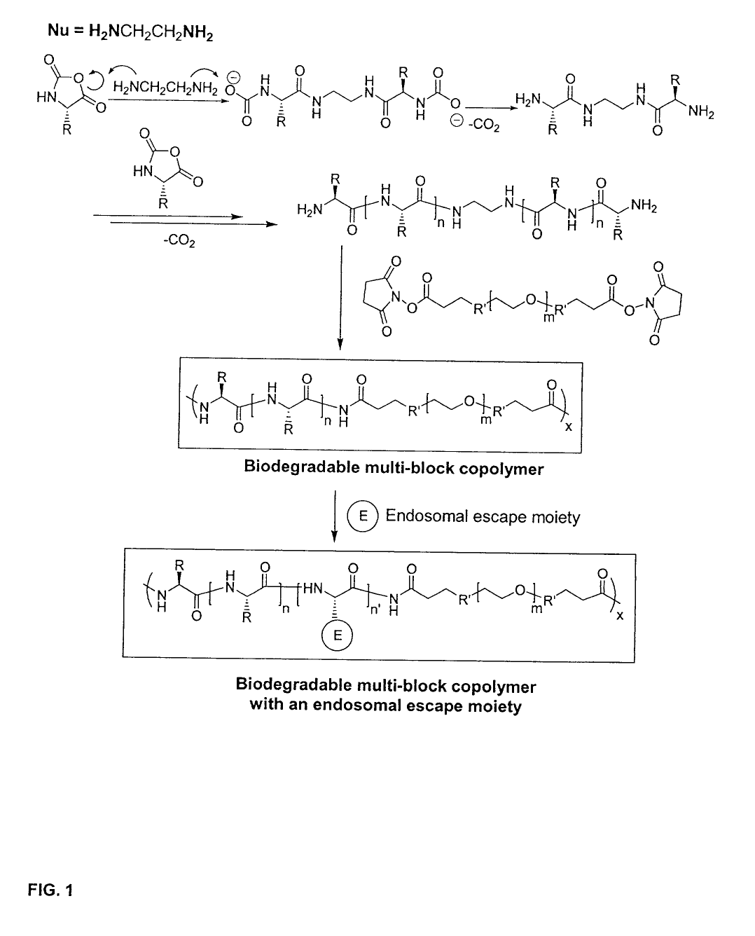

This example illustrates the preparation of biodegradable multi-block copolymers of poly(L-lysine) and PEG. The synthetic scheme is illustrated in FIG. 1.

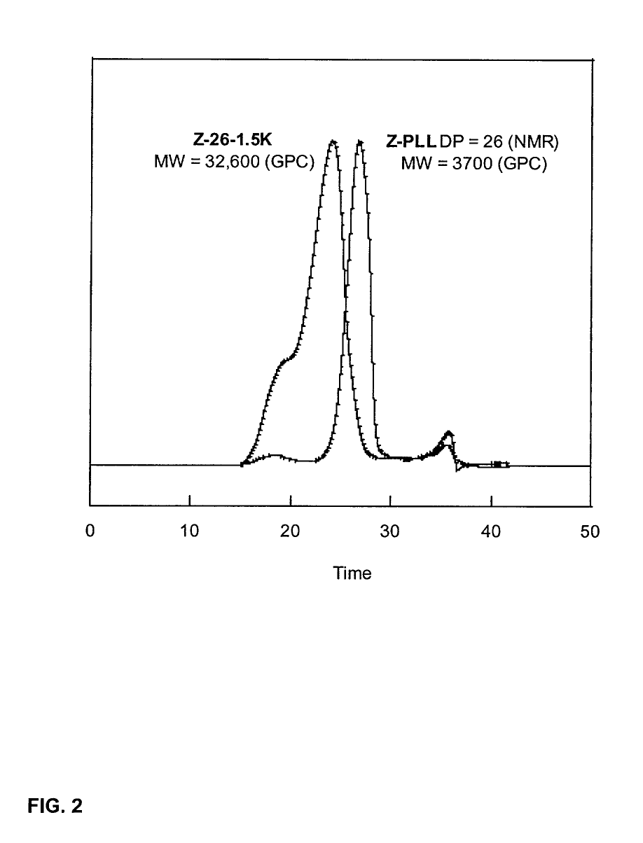

To a 250 ml flask under nitrogen atmosphere, equipped with a magnetic stirrer, were added 25 ml anhydrous dimethylformamide (DMF) and 5 g of N-carboxy-(N⁶-benzyloxycarbonyl)-L-lysine anhydride (Z-L-lysine NCA). Polymerization was initiated by the addition of a calculated amount of ethylenediamine by a microsyringe (corresponding to a molar Z-L-lysine NCA/initiator ratio of 10, 20 and 40). After stirring for 72 hours, a predetermined amount of a difunctional PEG, dissolved in a 25 ml anhydrous DMF, was added dropwise. The reaction mixture was condensed under reduced pressure after additional stirring for 72 hours. The condensed solution was precipitated into H₂O and the product was dried under vacuum overnight in the presence of P₂O₅. As shown in the GPC traces in FIG. 2, the shift of the peak to the higher molecular weight range clearly demonstrats that copolymer was successfully obtained. The results of copolymerization are listed in Table 1.

TABLE 1^{a}

| Run | | [M]ₒ/ | | | | M_{w}/ | |
|---|---|---|---|---|---|---|---|
| No. | Abbreviation | [I]ₒ | DP_{PLL}^{b} | DP_{PEG} | Mₙ (GPC)^{c} | Mₙ | x^{d} |
| 1 | 11-1.5K | 10 | 11 | 32 | 32,400 | 3.59 | 10 |
| 2 | 26-1.5K | 20 | 26 | 32 | 56,200 | 3.93 | 12 |
| 3 | 35-1.5K | 30 | 35 | 32 | 30,300 | 3.74 | 5 |
| 4 | 45-1.5K | 40 | 45 | 32 | 65,100 | 3.88 | 8 |

^{a}Polymerization at room temperature for 72 h before and after the addition of difunctional PEG.
^{b}Degree of polymerization by ¹H NMR.
^{c}Molecular weight measured by GPC with poly(L-lysine) standards.
^{d}Number of repeating units including PLL and PEG segment calculated based on the GPC results.

The obtained copolymer was then dissolved again in DMF for the deprotection reaction of the benzyloxycarbonyl group and added to a 250 ml flask, equipped with a magnetic stirrer and a dropping funnel. To a solution of the copolymer in 50 ml DMF was added 10 g of palladium catalyst. With vigorous stirring, 150 ml 95% formic acid was slowly added to the reaction mixture and the deprotection reaction was continued at room temperature for 14 hours. The palladium catalyst was filtered and washed with 150 ml 1N HCl to replace the formate salt by hydrochloric acid. The reaction mixture was condensed under reduced pressure to remove H₂O, precipitated into Et₂O and dried under vacuum overnight. The copolymers were then dissolved again in double distilled H₂O, centrifuged and filtered. The aqueous solution was freeze-dried for 2 days to obtain the biodegradable multi-block copolymers.

### EXAMPLE 2

This example illustrates the introduction of an endosomal escape moiety into the biodegradable multi-block copolymers of poly(L-lysine) and PEG. The synthetic scheme is illustrated in FIG. 1.

A predetermined amount of 1.3-dicyclohexylcarbodiimide, N-hydroxysuccinimide and an endosomal escape moiety, N,N-dimethyl-His-OH was dissolved in 5 ml anhydrous methyl sulfoxide (DMSO) and added to a 20 ml vial, equipped with a magnetic stirrer. After stirring for 2 hours, 200 mg of the biodegradable multi-block copolymer (Run No. 2) in 5 ml anhydrous DMSO was added to the vial for the conjugation of the endosomal escape moiety. The reaction was continued for an additional 12 hours at room temperature. The reaction mixture was centrifuged three times to remove the urea byproduct and precipitated into acetone. The solid was washed with Et₂O two times and dried under vacuum overnight. The conjugated copolymers were then dissolved again in double distilled H₂O, centrifuged and filtered. The aqueous solution was freeze-dried for 2 days to obtain biodegradable multi-block copolymers with an endosomal escape moiety.

### EXAMPLE 3

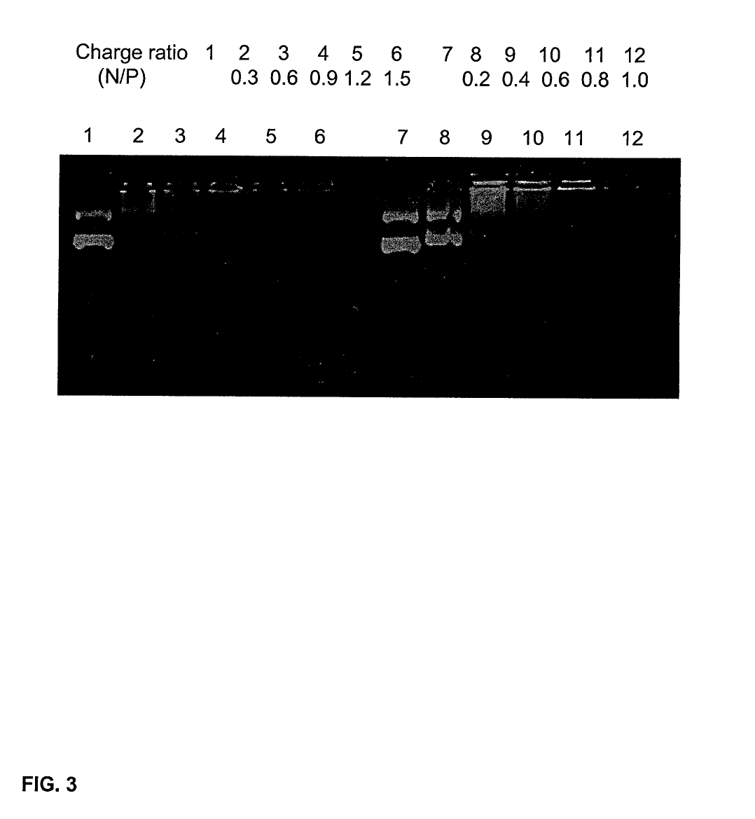

This example illustrates the preparation of a gene delivery composition, according to the present invention, by mixing a biodegradable multi-block copolymer and a pSV-β-gal plasmid DNA (e.g. Promega, Madison, Wis.) in PBS buffer. The biodegradable multi-block copolymer utilized consisted of PLL (degree of polymerization, 11, 26 and 45) and PEG (molecular weight, 1,500) and was prepared as described in Example 1. To study the effect of charge ratio on gene transfer, the plasmid and the biodegradable multi-block copolymer complexes were prepared at charge ratios of 0.3, 0.6, 0.9, 1.2, 1.5, 1.8, 2.1 and 2.4. The control composition contained only the 25,700 molecular weight PLL homopolymer instead of the copolymer. Stable complexes were formed with the copolymer and the aqueous plasmid DNA solution based on the fact that no precipitation or aggregation was observed at wide concentration ranges of the complexes in the PBS buffer. The complex formation of the plasmid DNA and the cationic copolymer was tested by agarose gel electrophoresis and the results are shown in FIG. 3. As depicted in FIG. 3, complete neutralization was achieved at the charge ratios of pSV-β-gal plasmid/copolymers from 0.9 to 1.2

### EXAMPLE 4

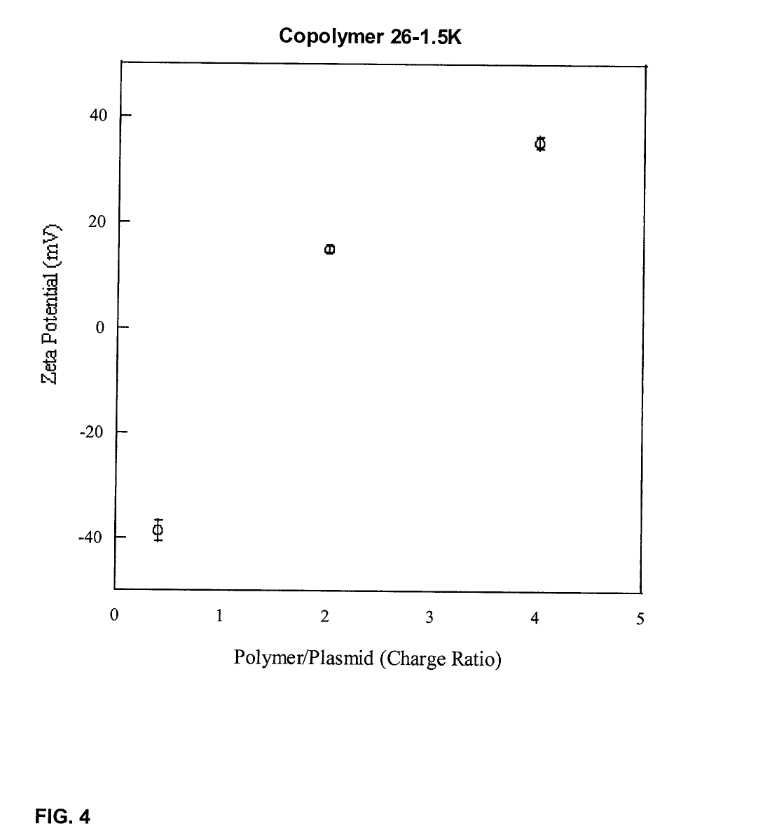

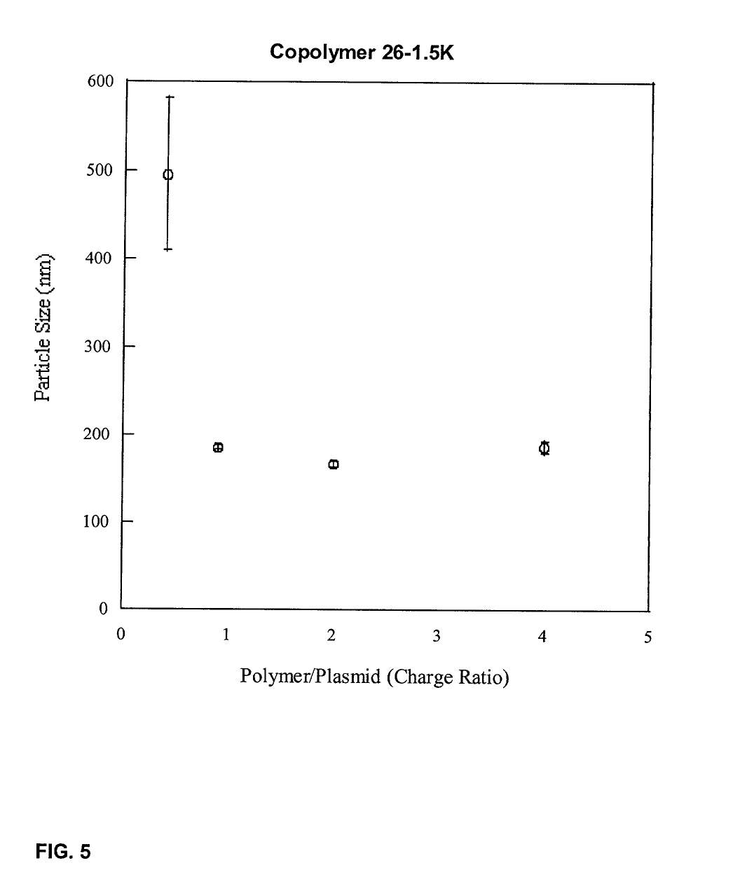

In this example, the zeta potential and particle size of copolymers and plasmid DNA, according to the present invention, were measured by a zetapotentiometer. Complexes different in the composition of the copolymer and plasmid DNA were prepared in double distilled H₂O and diluted to 4 ml as the final volume. The sample was subjected to the measurement of zeta potential and mean particle size by a BI-MAS (Brookhaven Instruments Co.) at 25° C., a wavelength of 677 nm, and with the constant angle of 90°. Zeta potential measurement, FIG. 4, also confirmed the results of the gel retardation assy. The copolymer (Run No. 2 in Table 1) was employed to prepare a complex based on a charge ratio between the copolymer and DNA of from 0.5 to 4. Complete neutralization was around the charge ratio of 1. Using the same instrument, the particle size of the complexes was estimated. In the range of the compositions where the amount of copolymer was not enough to effectively condense the plasmid, a particle size of over 400 nm was obtained. After complete neutralization over the charge ratio of 1, particle sizes ranged from 167.0±3.7 to 187.1±6.8 nm and reached an almost constant value around 180 nm as shown in FIG. 5.

### EXAMPLE 5

This example illustrates the evaluation of copolymer cytotoxicity performed by the MTT assay, as originally described by T. Mosmann, Rapid Colorimetric Assay for Cellular Growth and Survival: Application to Proliferation and Cytotoxicity Assays, 65 J. Immunol Methods 55-63 (1983).

The cytotoxicity of the copolymers (Run No. 2) of the present invention was compared to a BPS buffer control and a PLL with a molecular weight of 25,700, which is the PLL polymer most commonly used for gene delivery application. 293T cells were seeded at a cell density of 4.5×10⁴ cells/well in 24-well multiwell plates (Falcon Co., Becton Dickenson, Franklin Lakes, N.J.) and incubated for 24 hours. Plasmid/copolymer complex (1 μg plasmid/copolymer corresponding to the charge ratio) was added in the absence of fetal bovine serum and incubated for 4 hours at 37° C. in 5% CO₂. At the end of the transfection experiment, the transfection mixture was replaced with 350 μl fresh DMEM medium with 10% fetal bovine serum and the cells were incubated for an additional 24 hours. 50 μl MTT solution (5 mg/ml) in PBS buffer was added and the plates were incubated for 4 hours at 37° C. MTT containing medium was removed and 450 μl DMSO was added to dissolve the formazan crystal formed by the live cells. Absorbance was measured at 570 nm. The cell viability (%) was calculated according to the following equation;

Cell viability(%)=(OD₅₇₀₍ₛₐₘₚₗₑ₎/OD_{570(control)})×100

where OD₅₇₀₍ₛₐₘₚₗₑ₎ represents the measurement from the wells treated with copolymer and OD_{570(control)} from the wells treated with PBS buffer only.

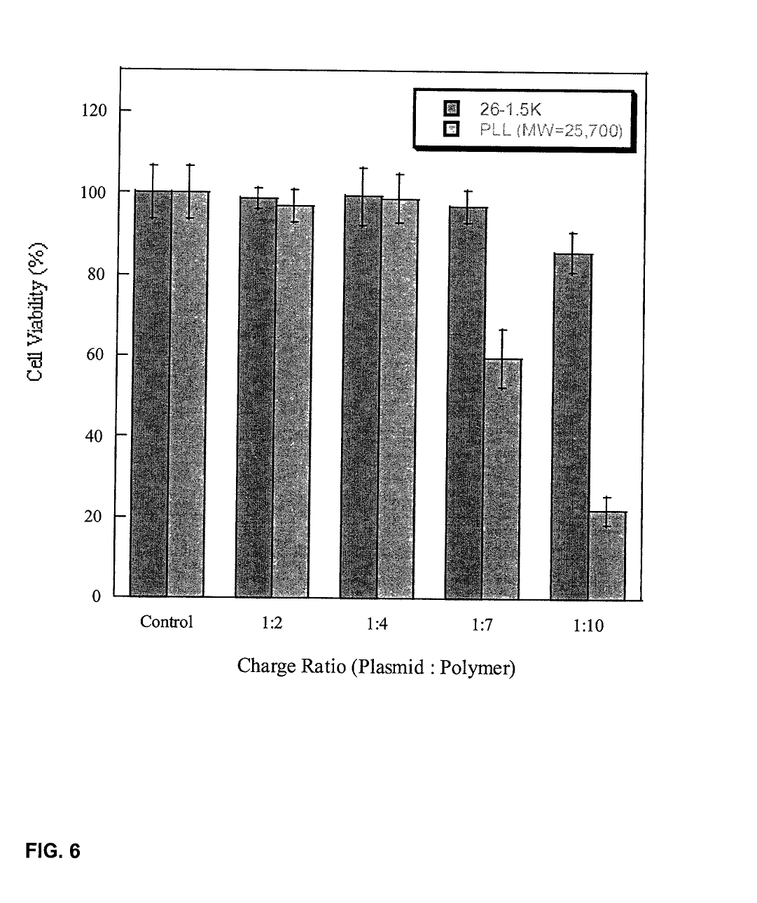

Decreased cytotoxicity of the present copolymers is confirmed in FIG. 6 showing that cell viability of over 90% was obtained for the all the employed charge ratios, while that of PLL with a molecular weight of 25,700 drastically decreased down to 20% at a charge ratio of 1:10.

### EXAMPLE 6

In this example, compositions comprising pSV-β-gal plasmid DNA and the copolymer (Run No. 2) in a charge ratio between 1:2 and 1:10 were prepared and tested for the in vitro delivery and expression of pSV-β-gal plasmid DNA in the 293T cell line. The plasmid pSV-β-gal (EMBL accession no. X65335) is a positive control vector for monitoring transfection efficiencies of mammalian cells. Cell extracts of transfected cells can be measured directly for β-galactosidase activity by a spectrophotometric assay.

In vitro transfection of the 293T cells was performed in 6-well plates seeded at a cell density of 2.25×10⁵ cells/well 24 hours prior to the addition of transfection compositions. The copolymer pSV-β-gal composition (6 μg plasmid/copolymer corresponding to the charge ratio) was added to cells in the absence of 10% fetal bovine serum. Serum-free transfection mixtures were incubated for 4 hours, followed by supplementation with fetal bovine serum to a level of 10%. Cells were incubated for 40 to 48 hours in an incubator at 37° C. in 5% CO₂ and then lysed by addition of Promega Reporter Lysis Buffer (cat. No. E3971). The β-galactosidase activity in the transfected cell lysates was measured by absorbance at 415 nm.

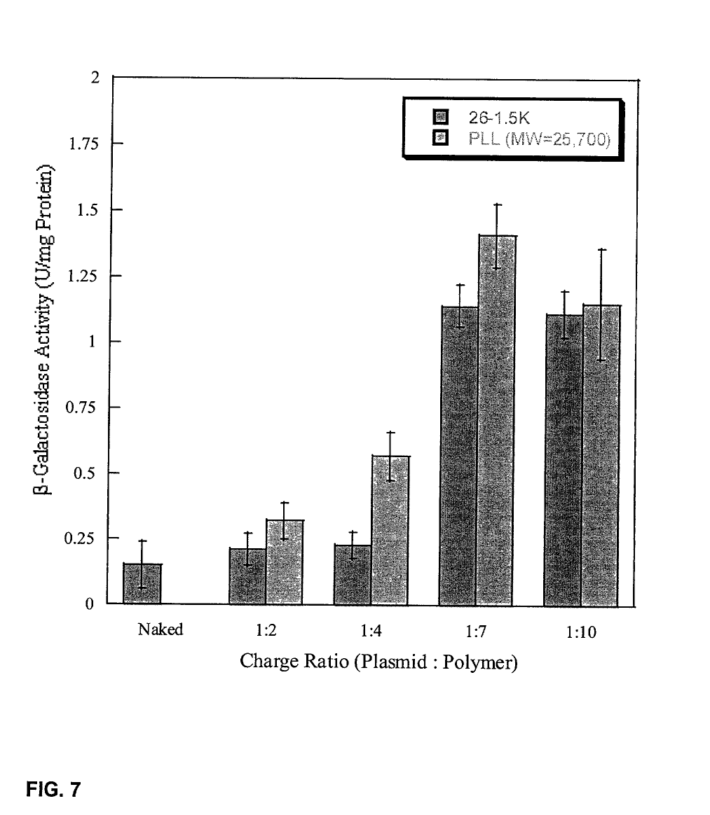

FIG. 7 shows the relative β-galactosidase activity of the composition according to the present invention as compared to a PLL control with a molecular weight of 25,700. The transfection efficiency, as measured by β-galactosidase activity of transfected cell extracts, reached a maximun value at a charge ratio of 1:7 and decreased as the charge ratio of the plasmid to the copolymer was raised. It is the important feature of the biodegradable multi-block copolymers of the present invention that cytotoxicity towards cells is negligible at the concentrations required for optimal transfection.

### EXAMPLE 7

This example illustrates the characterization of biodegradable multi-block copolymers of poly(L-lysine) and PEG. Molecular weight and degradation under physiological conditions were investigated using gel permeation chromatography (GPC).

50 mg multi-block copolymer was dissolved in 20 mL PBS buffer at pH of 7.4 and the aqueous solution was incubated at 37° C. At an appropriate time interval, 1 mL aliquot was taken and applied to Shimatsu Gel Permeation Chromatography for the measurement of molecular weight. Double distilled water with 0.1 vol % trifluoroacetic acid was used as a solvent at a flow rate of 1.0 mL/min.

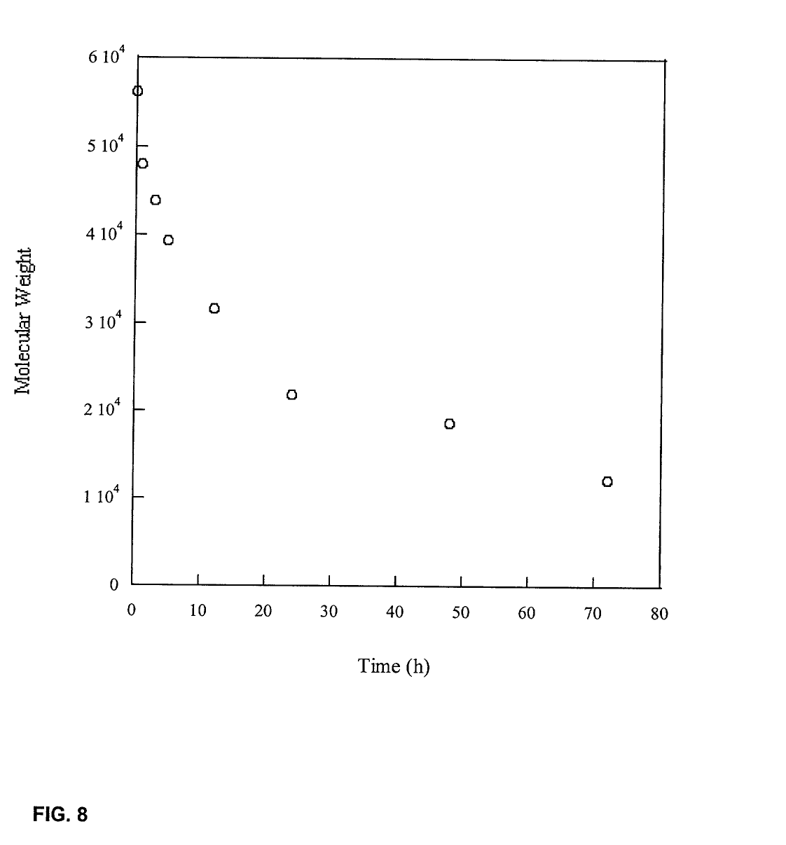

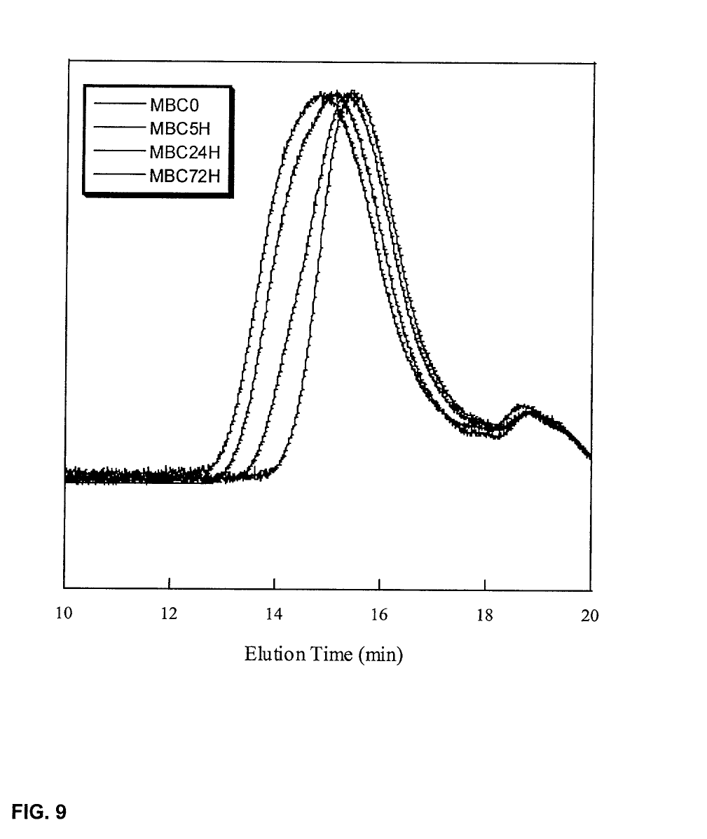

FIG. 8 shows that the synthesized multi-block copolymers were biodegradable under physiological conditions (pH 7.4, 37° C.). Biodegradability test under physiological condition revealed that the molecular weight of copolymers was decreased to the 20% of initial molecular weight within 72 h. The representative GPC traces of the degraded copolymers for 5, 24, 72 h are illustrated in FIG. 9.

### EXAMPLE 8

In this example, compositions comprising pSV-β-gal plasmid DNA and the copolymers at a charge ratio of 1:7 were prepared and tested for the expression of pSV-β-gal plasmid DNA in the 293T cell line in the presence and absence of fetal bovine serum. Cell extracts of transfected cells can be measured directly for β-galactosidase activity by a spectrophotometric assay.

In vitro transfection was performed in 6-well plates which were seeded with the 293T cells at a cell density of 2.25×10⁵ cells/well 24 hours prior to the addition of transfection compositions. The copolymer pSV-β-gal composition (6 μg plasmid/copolymer corresponding to the charge ratio) was added to cells in the presence and the absence of 10% fetal bovine serum. Both transfection mixtures were incubated for 4 hours, followed by supplementation with fetal bovine serum to a level of 10%. Cells were incubated for 40 to 48 hours in an incubator at 37° C. in 5% CO₂ and then lysed by addition of Promega Reporter Lysis Buffer (cat. No. E3971). The β-galactosidase activity in the transfected cell lysates was measured by the absorbance at 415 nm.

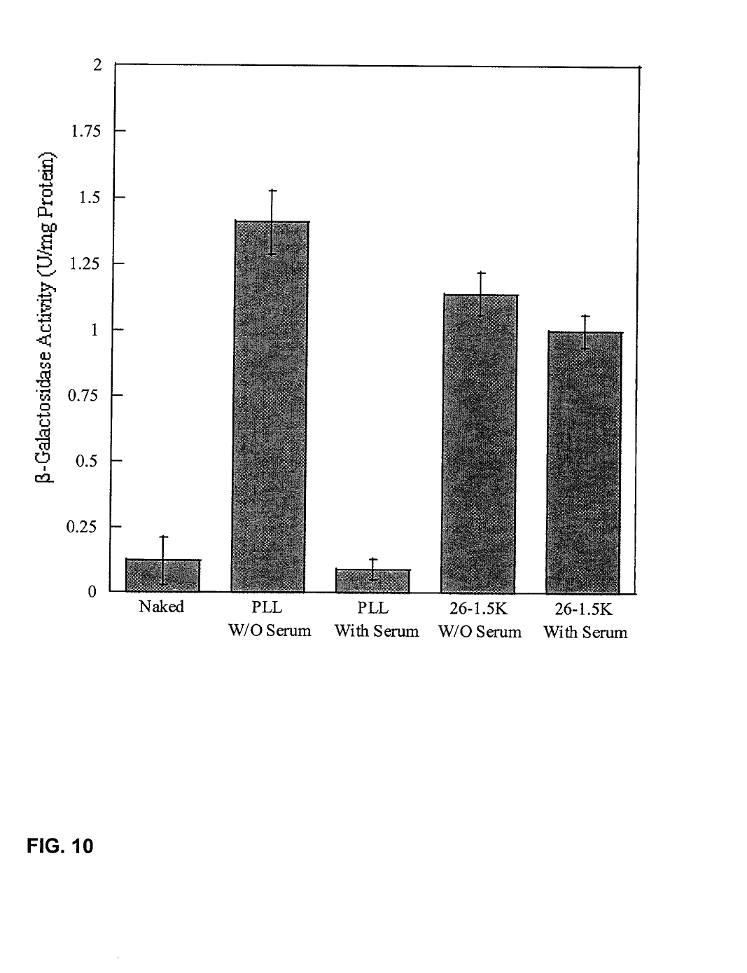

FIG. 10 shows the relative β-galactosidase activity of the composition according to the present invention as compared to a PLL control with a molecular weight of 25,700. The transfection efficiencies of the copolymers were not significantly affected by the presence or absence of serum, while that of a PLL control decreased to the same level as naked genes in the presence of serum.

### EXAMPLE 9

This example illustrates the evaluation of the cytotoxicity of copolymers conjugated with an endosomal escape moiety performed by an MTT assay, as originally described by T. Mosmann, Rapid Colorimetric Assay for Cellular Growth and Survival: Application to Proliferation and Cytotoxicity Assays, 65 J. Immunol Methods 55-63 (1983).

The cytotoxicity of the copolymers (Run No. 2) of the present invention was compared to a BPS buffer control, a PLL with the molecular weight of 25,700, which is the PLL polymer most commonly used for gene delivery applications, and copolymers without conjugation with an endosomal escape moiety. 293T cells were seeded at a cell density of 4.5×10⁴ cells/well in 24-well multi-well plates (Falcon Co., Becton Dickenson, Franklin Lakes, N.J.) and incubated for 24 hours. Plasmid/copolymer complex (1 μg plasmid/copolymer corresponding to a charge ratio of 1:7 plasmid:copolymer) was added in the absence of fetal bovine serum and incubated for 4 hours at 37° C. in 5% CO₂. At the end of the transfection experiment, the transfection mixture was replaced with 350 μl fresh DMEM medium with 10% fetal bovine serum and the cells were incubated for additional 24 hours. 50 μl of MTT solution (5 mg/ml) in PBS buffer was added and the plates were incubated for 4 hours at 37° C. The MTT containing medium was removed and 450 μl DMSO was added to dissolve the formazan crystal formed by the live cells. Absorbance was measured at 570 nm. The cell viability (%) was calculated according to the following equation;

Cell viability(%)=(OD₅₇₀₍ₛₐₘₚₗₑ₎/OD_{570(control)})×100

where OD₅₇₀₍ₛₐₘₚₗₑ₎ represents the measurement from the wells treated with copolymer and OD_{570(control)} from the wells treated with PBS buffer only.

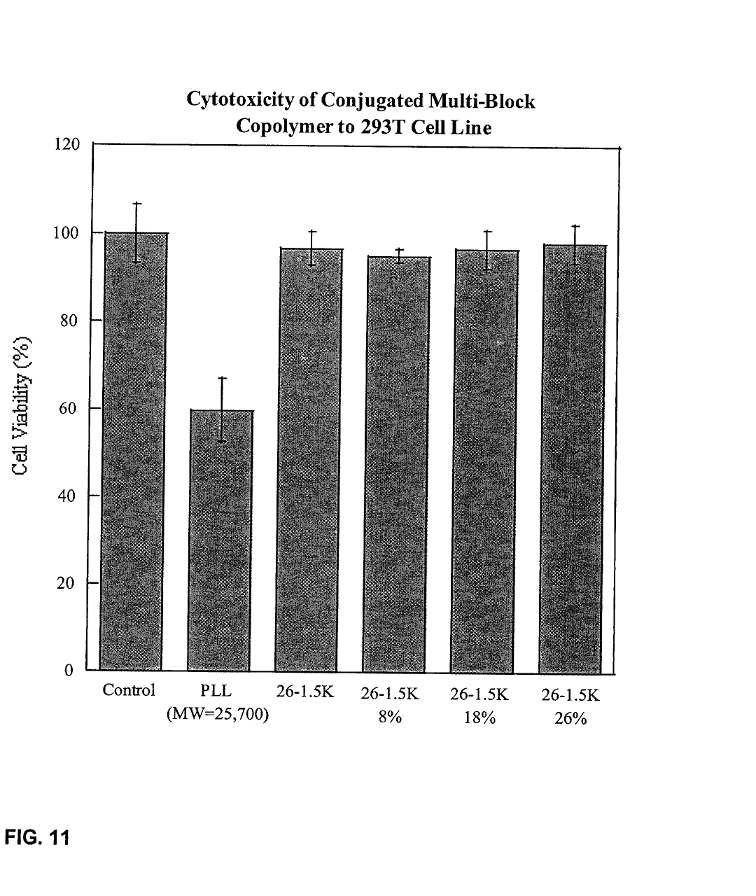

As shown in FIG. 11, the cytotoxicity of the copolymers conjugated with different amounts of endosomal escape moieties was independent of the amount of conjugation and almost negligible showing a cell viability of over 95%, while that of PLL with a molecular weight of 25,700 at the given charge ratio was slightly less than 60%.

### EXAMPLE 10

In this example, compositions comprising pSV-β-gal plasmid DNA and the copolymers conjugated with an endosomal escape moiety at a charge ratio of 1:7 were prepared and tested for the in vitro delivery and expression of pSV-β-gal plasmid DNA in the 293T cell line. The plasmid pSV-β-gal (EMBL accession no. X65335) is a positive control vector for monitoring transfection efficiencies of mammalian cells. Cell extracts of transfected cells can be measured directly for β-galactosidase activity by a spectrophotometric assay.

In vitro transfection was performed in 6-well plates which were seeded with the 293T cells at a cell density of 2.25×10⁵ cells/well 24 hours prior to the addition of transfection compositions. The copolymer pSV-β-gal composition (6 μg plasmid/copolymer corresponding to the charge ratio) was added to cells in the absence of 10% fetal bovine serum. Serum-free transfection mixtures were incubated for 4 hours, followed by supplementation with fetal bovine serum to a level of 10%. The cells were incubated for 40 to 48 hours in an incubator at 37° C. in 5% CO₂ and then lysed by addition of Promega Reporter Lysis Buffer (cat. No. E3971). The β-galactosidase activity in the transfected cell lysates was measured by the absorbance at 415 nm.

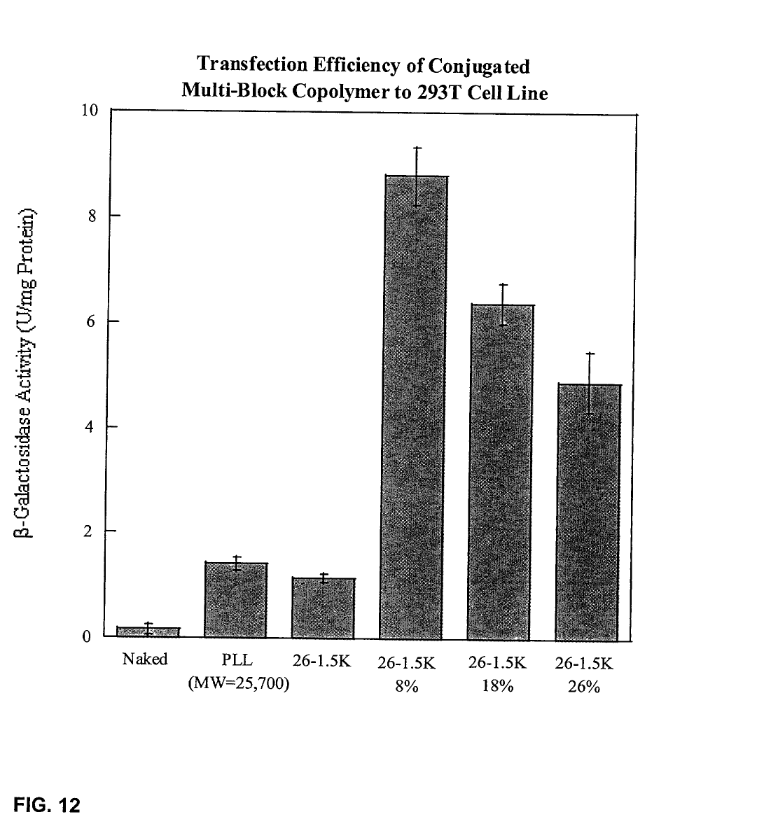

FIG. 12 shows the relative β-galactosidase activity of the composition according to the present invention as compared to a PLL control with a molecular weight of 25,700 and the copolymer without conjugation with an endosomal escape moiety. The transfection efficiency, as measured by β-galactosidase activity of transfected cell extracts, reached a maximun value at 8 mol % of conjugation of the endosomal escape moiety to the copolymer and decreased as the molar conjugation of the endosomal escape moiety to the copolymer increased up to 26%.

### EXAMPLE 11

In this example, compositions comprising pSV-β-gal plasmid DNA and the copolymers conjugated with an endosomal escape moiety at a charge ratio of 1:7 were prepared and tested for the expression of pSV-β-gal plasmid DNA in the 293T cell line in the presence and absence of fetal bovine serum. Cell extracts of transfected cells can be measured directly for β-galactosidase activity by a spectrophotometric assay.

In vitro transfection was performed in 6-well plates which were seeded with the 293T cells at a cell density of 2.25×10⁵ cells/well 24 hours prior to the addition of transfection compositions. The copolymer pSV-β-gal composition (6 μg plasmid/copolymer corresponding to the charge ratio) was added to cells in the absence and the presence of 5%, 10% and 20% fetal bovine serum. Both transfection mixtures were incubated for 4 hours, followed by supplementation with fetal bovine serum to a level of 10%. Cells were incubated for 40 to 48 hours in an incubator at 37° C. in 5% CO₂ and then lysed by addition of Promega Reporter Lysis Buffer (cat. No. E3971). The β-galactosidase activity in the transfected cell lysates was measured by the absorbance at 415 nm.

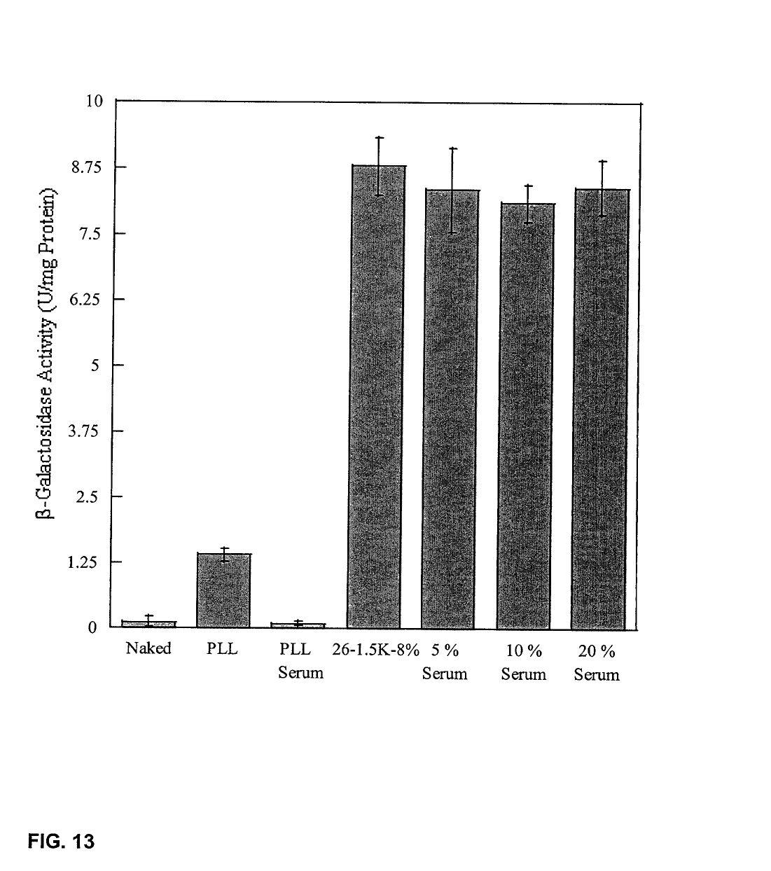

FIG. 13 shows the relative β-galactosidase activity of the composition according to the present invention as compared to a PLL control with a molecular weight of 25,700. The transfection efficiencies of the copolymers conjugated with an endosomal escape moiety were not significantly influenced by the presence or absence of serum, while that of a PLL control decreased to the same level as naked genes in the presence of serum.

Thus, among the various embodiments taught there has been disclosed a composition comprising a novel biodegradable multi-block copolymer of PAA and a hydrophilic polymer and method of use thereof for delivering bioactive agents, such as DNA, RNA, oligonucleotides, proteins, peptides, and drugs. It will be readily apparent to those skilled in the art that various changes and modifications of an obvious nature may be made without departing from the spirit of the invention, and all such changes and modifications are considered to fall within the scope of the invention as defined by the appended claims.

We claim:

1. A biodegradable multi-block copolymer comprising a poly(amino acid) (PAA), and a hydrophilic polymer, wherein the PAA is linked with the hydrophilic polymer by a biodegradable linkage.

2. The biodegradable multi-block copolymer of claim 1 wherein the molar ratio of the PAA to the hydrophilic polymer is within a range of 0.5:1 to 2:1.

3. The biodegradable multi-block copolymer of claim 1 wherein the biodegradable linkage is a member selected from the group consisting of esters, amides, urethanes and carbonate.

4. The biodegradable multi-block copolymer of claim 1 wherein the PAA has an average molecular weight of 800 to 200,000 Daltons, the hydrophilic polymer has an average molecular weight of 500 to 20,000 Daltons.

5. The biodegradable multi-block copolymer of claim 1, wherein the hydrophilic polymer is a member selected from the group consisting of polyethylene glycol (PEG), poloxamers, poly(acrylic acid), poly(styrene sulfonate), carboxymethylcellulose, poly(vinyl alcohol), polyvinylpyrrolidone, alpha-substituted poly(oxyalkyl) glycols, poly(oxyalkyl) glycol copolymers and block copolymers, and activated derivatives thereof.

6. The biodegradable multi-block copolymer of claim 5, wherein the hydrophilic polymer is polyethylene glycol (PEG).

7. The biodegradable multi-block copolymer of claim 1, wherein the PAA has a high proportion of positively charged side groups and is a member selected from the group consisting of polylysine, polyarginine, block and graft copolymers thereof and activated derivatives thereof.

8. The biodegradable multi-block copolymer of claim 7, wherein the PAA is polylysine, polyarginine, block and graft copolymers thereof, or activated derivatives thereof.

9. The biodegradable multi-block copolymer of claim 1 further comprising a targeting moiety selected from the group consisting of transferrin, asialoglycoprotein, antibodies, antibody fragments, low density lipoproteins, interleukins, GM-CSF, G-CSF, M-CSF, stem cell factors, erythropoietin, epidermal growth factor (EGF), insulin, asialoorosomucoid, mannose-6-phosphate, mannose, Lewis^{X} and sialyl Lewis^{X}, N-acetyllactosamine, galactose, lactose, thrombomodulin, fusogenic agents such as polymixin B and hemaglutinin HA2, lysosomotrophic agents, and nucleus localization signals (NLS).

10. The biodegradable multi-block copolymer of claim 1 further comprising an endosomal escape moiety with buffering capacities between pH 4.0 and 7.2.

11. The biodegradable multi-block copolymer of claim 10 wherein the endosomal escape moiety is a member selected from the group consisting of imidazole derivatives, histidine derivatives, poly(ethylenimine) and poly(L-histidine).

12. A biodegradable multi-block copolymer comprising a poly(amino acid) (PAA) and polyethylene glycol (PEG), wherein the PAA is linked with a PEG by a biodegradable linkage selected from the group consisting of esters, amides, urethanes and carbonate.

13. The biodegradable multi-block copolymer of claim 12, wherein the PAA has an average molecular weight of 800 to 200,000 Daltons, the PEG has an average molecular weight of 500 to 20,000 Daltons, and the molar ratio of the PAA to the PEG is within a range of 0.5:1 to 2:1.

14. The biodegradable multi-block copolymer of claim 12, wherein the PAA has a high proportion of positively charged side groups and is a member selected from the group consisting of polylysine, polyarginine, block and graft copolymers thereof and activated derivatives thereof.

15. The biodegradable multi-block copolymer of claim 12 further comprising a targeting moiety selected from the group consisting of transferrin, asialoglycoprotein, antibodies, antibody fragments, low density lipoproteins, interleukins, GM-CSF, G-CSF, M-CSF, stemcell factors, erythropoietin, epidermal growth factor (EGF), insulin, asialoorosomucoid, mannose-6-phosphate, mannose, Lewis^{X} and sialyl Lewis^{X}, N-acetyllactosamine, galactose, lactose, and thrombomodulin, fusogenic agents such as polymixin B and hemaglutinin HA2, lysosomotrophic agents, and nucleus localization signals (NLS).

16. The biodegradable multi-block copolymer of claim 12 further comprising an endosomal escape moiety selected from a compound or polymer with buffering capacities between pH 4.0 and 7.2.

17. The biodegradable multi-block copolymer of claim 12 wherein the endosomal escape moiety is a member selected from the group consisting of imidazole derivatives, histidine derivatives, poly(ethylenimine) and poly(L-histidine).

18. A biodegradable multi-block copolymer represented by the formula: 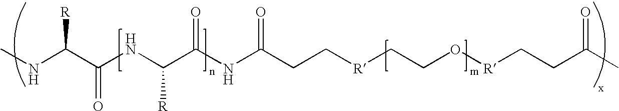
wherein n is an integer from 5 to 1,000, m is an integer from 10 to 500, x is an integer from 1 to 100, R′ represent a biodegradable linkage, and R represents the residual portion of an amino acid or derivatives thereof.

19. The biodegradable multi-block copolymer of claim 18 wherein R′ is a linkage member selected from the group consisting of ester, amide, urethane and carbonate and R represents the residual portion of an amino acid or derivatives thereof which is positively charged under a physiological condition such as lysine, arginine or derivatives thereof.

20. The biodegradable multi-block copolymer of claim 18 wherein R′ is a linkage member selected from the group consisting of ester, amide, and urethane R is a residual portion of a positively charged amino acid selected from the group consisting of lysine, arginine, block and graft cop olymers thereof and activated derivatives thereof.

21. The biodegradable multi-block copolymer of claim 18 further comprising an endosomal escape moiety selected from the group consisting of imidazole derivatives, histidine derivatives, poly(ethylenimine) and poly(L-histidine).

22. A composition for the safe and efficient delivery system of bioactive agents comprising a biodegradable multi-block copolymer capable of forming a stable complex with a bioactive agent, said biodegradable multi-block copolymer comprising a poly(amino acid) (PAA), and a hydrophilic polymer, wherein the PAA is linked with the hydrophilic polymer by a biodegradable linkage.

23. A composition for the safe and efficient delivery system of bioactive agents comprising a biodegradable multi-block copolymer capable of forming a stable complex with a bioactive agent, said biodegradable multi-block copolymer comprising a poly(amino acid) (PAA), and polyethylene glycol (PEG), wherein the PAA is linked with the hydrophilic polymer by a biodegradable linkage selected from the group consisting of ester, amide and urethane.

24. A transfection formulation comprising a biodegradable multi-block copolymer complexed with a selected nucleic acid in the proper charge ratio(positve charge of the copolymer/negative charge of the nucleic acid) that is optimally effective for both in vivo and in vitro transfection, said biodegradable multi-block copolymer comprising a poly(amino acid) (PAA), and a hydrophilic polymer, wherein the PAA has a high proportion of positively charged side groups and is a member selected from the group consisting of polylysine, polyarginine, block and graft copolymers thereof and activated derivatives thereof; and the PAA is linked with the hydrophilic polymer by a biodegradable linkage.

25. The formulation of claim 24, wherein the hydrophilic polymer is polyethylene glycol (PEG).

26. The formulation of claim 25, wherein the PAA has an average molecular weight of 800 to 200,000 Daltons, the PEG has an average molecular weight of 500 to 20,000 Daltons, and the molar ratio of the PAA to the PEG is within a range of 0.5:1 to 2:1.

27. The formulation of claim 24, wherein the weight ratio of DNA to the biodegradable multi-block copolymer is preferably within a range of 1:0.3 to 1:16.

28. The formulation of claim 24 further comprising a targeting moiety selected from the group consisting of transferrin, asialoglycoprotein, antibodies, antibody fragments, low density lipoproteins, interleukins, GM-CSF, G-CSF, M-CSF, stemcell factors, erythropoietin, epidermal growth factor (EGF), insulin, asialoorosomucoid, mannose-6-phosphate, mannose, Lewis^{X} and sialyl Lewis^{X}, N-acetyllactosamine, galactose, lactose, and thrombomodulin, fusogenic agents such as polymixin B and hemaglutinin HA2, lysosomotrophic agents, and nucleus localization signals (NLS).

29. The formulation of claim 24 further comprising an endosomal escape moiety selected from the group consisting of imidazole derivatives, histidine derivatives, poly(ethylenimine) and poly(L-histidine).

30. A method of transfecting a cell in vitro with biodegradable water soluble multi-block copolymers and a selected plasmid DNA, comprising the steps of:
(a) providing a formulation comprising a biodegradable multi-block copolymer complexed with a selected nucleic acid in the proper charge ratio, said biodegradable multi-block copolymer comprising a poly(amino acid) (PAA), and a hydrophilic polymer, wherein the PAA has a high proportion of positively charged side groups and is a member selected from the group consisting of polylysine, polyarginine, block and graft copolymers thereof and activated derivatives thereof; and the PAA is linked with the hydrophilic polymer by a biodegradable linkage,
(b) contacting the cell with an effective amount of the formulation such that the cell internalizes the selected nucleic acid; and
(c) culturing the cell with the internalized selected plasmid DNA under conditions favorable for the growth thereof.

31. The method of claim 30, wherein the hydrophilic polymer is a polyethylene glycol (PEG).

32. The method of claim 30, wherein the PAA has an average molecular weight of 800 to 1,000,000 Daltons, the PEG has an average molecular weight of 500 to 20,000 Daltons, and the molar ratio of the PAA to the PEG is within a range of 0.5:1 to 2:1.

33. The method of claim 30, wherein the weight ratio of DNA to the biodegradable multi-block copolymer is preferably within a range of 1:0.3 to 1:16.

34. The method of claim 30 further comprising a targeting moiety selected from the group consisting of transferrin, asialoglycoprotein, antibodies, antibody fragments, low density lipoproteins, interleukins, GM-CSF, G-CSF, M-CSF, stemcell factors, erythropoietin, epidermal growth factor (EGF), insulin, asialoorosomucoid, mannose-6-phosphate, mannose, Lewis^{X} and sialyl Lewis^{X}, N-acetyllactosamine, galactose, lactose, and thrombomodulin, fusogenic agents such as polymixin B and hemaglutinin HA2, lysosomotrophic agents, and nucleus localization signals (NLS).

35. The method of claim 30 further comprising an endosomal escape moiety selected from the group consisting of imidazole derivatives, histidine derivatives, poly(ethylenimine) and poly(L-histidine).
